(19) United States
(12) Patent Application Publication
(10) Pub. No.: US 2010/0087405 A1
(43) Pub. Date: Apr. 8, 2010

(54) FOOD SUPPLEMENTATION COMPOSITION CONTAINING ONE OR MORE VITAMIN D3 COMPOUNDS AND ONE OR MORE MAGNESIUM SALTS

(76) Inventors: Robert Elliott, Queensland (AU); Gilbert Weber, Magden (CH)

Correspondence Address: NIXON & VANDERHYE, PC, 901 NORTH GLEBE ROAD, 11TH FLOOR, ARLINGTON, VA, 22203, US

(21) Appl. No.: 12/440,788

(22) PCT Filed: Sep. 14, 2007
(86) PCT No.: PCT/EP07/07996

§ 371 Date: Nov. 6, 2009

(30) Foreign Application Priority Data
Sep. 14, 2006 (EP) 06019231.7

(51) Int. Cl. A61K 31/59; C07C 403/00

(52) U.S. Cl. 514/167; 552/653

(57) ABSTRACT

Use of one or more vitamin D₃ compounds chosen from the group 1-α-hydroxy vitamin D₃, 25-hydroxy vitamin D₃, 1-α-,25-dihydroxy vitamin D₃, in combination with one or more magnesium salts to prevent milk fever in animals.

The present invention relates to a food supplementation composition containing one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts. More particularly, the invention relates to a food supplementation composition containing the above mentioned ingredients for the prevention of milk fever in animals, particularly in dairy cows.

The invention further relates to the use of one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts, especially magnesium chloride and/or magnesium sulphate, in the manufacture of a food or veterinary composition for the prevention of milk fever in animals, particularly dairy cows.

In another aspect, the invention relates to a method for the prevention of milk fever in animals, particularly dairy cows, which comprises administering to an animal in need of such treatment one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts, especially magnesium chloride and/or magnesium sulphate.

The transition between late pregnancy and early lactation (3-4 weeks post partum) is a high risk period for many metabolic disorders in dairy cows. In particular the failure to maintain blood calcium concentrations after calving due to the rapid demand for colostrum production can lead to both hypocalcaemia and clinical milk fever.

Dairy cows are normally fed on predominantly forage based rations before calving and the resulting high potassium intakes can result in metabolic alkalosis. In order to prepare the cow for calving, anionic supplements such as magnesium chloride or magnesium sulphate are normally included as part of a pre-calving supplementation programme.

In accordance with the present invention it has now been found that the above mentioned problems can be eliminated or substantially ameliorated by administering to the animals an effective amount of one or more vitamin D₃ compounds chosen from the group 1-α-hydroxy vitamin D₃, 25-hydroxy vitamin D₃, 1-α-,25-dihydroxy vitamin D₃, in combination with one or more magnesium salts, especially magnesium chloride and/or magnesium sulphate.

A food supplementation composition containing one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts is therefore one preferred embodiment of the present invention.

According to the present invention one or more vitamin D₃ compounds and one or more magnesium salts are suitably administered together with the food. Food may be supplemented by admixing one or more of the vitamin D₃ compounds according to the present invention—e.g. 25-hydroxy vitamin D₃ as a commercial formulation such as available under the Trademark ROVIMIX® Hy-D® 1.25%—and one or more magnesium salts to regular food. The terms “food” or “food composition” as used herein comprise solid and liquid food as well as drinking fluids such as drinking water.

A dry food composition for dairy cows according to the present invention contains, if it is used as the sole vitamin D₃ compound, 25-hydroxy vitamin D₃ preferably in an amount of 100 μg per kg of the dry food composition to 1000 μg per kg of the dry food composition, more preferably in an amount of 250 μg per kg of the dry food composition to 750 μg per kg of the dry food composition, most preferred about 500 μg per kg of the dry food composition.

As 1-α-hydroxy vitamin D₃ and 1-α-, 25-dihydroxy vitamin D₃ are both approximately 10 times more active than 25-hydroxy vitamin D₃, their amount in a dry food composition should be adapted accordingly. The same applies for mixtures of two or three of the vitamin D₃ compounds according to the present invention. It is most preferred to use 25-hydroxy vitamin D₃ as the sole vitamin D₃ compound.

The food composition further comprises from 5 g per kg of the dry food composition to 15 g per kg of the dry food composition of one or more magnesium salts, especially magnesium chloride and/or magnesium sulphate. Magnesium chloride is most preferred.

According to the present invention it is further advantageous if the composition also contains one or more of the following ingredients: Vitamin A, Vitamin E, Biotin, copper (e.g. as CuSO₄), zinc (e.g. as ZnSO₄), cobalt (e.g. as CoSO₄), selenium (e.g. as Na₂SeO₃), iodine (e.g. as KI), manganese (e.g. as MnSO₄) and/or calcium (e.g. as CaSO₄).

It is preferred to use calcium sulphate as calcium carrier in a composition according to the present invention.

In a further preferred embodiment of the present invention the composition is a premix, i.e. one or more vitamin D₃ compounds according to the present invention together with one or more magnesium salts are—e.g. as a formulated powder—added to other minerals, vitamins, amino acids and/or trace elements in a higher concentration in order to form the premix. For use the premix is added to and thoroughly mixed with a regular animal food to achieve even distribution therein.

A premix according to the present invention may be prepared by adding the active ingredients to regular food components in a concentration of from about 5 mg per kg of the premix to about 50 mg per kg of the premix of 25-hydroxy vitamin D₃—if 25-hydroxy vitamin D₃ is the sole vitamin D₃ compound—and from about 250 g per kg of the premix to about 750 g per kg of the premix of one or more magnesium salts, especially magnesium chloride and/or magnesium sulphate. If 2 kg of such premix are added per 100 kg of regular food this typically meets the individual need of the animal by normal food consumption.

When the composition is prepared in the form of a premix the premix preferably comprises from 3 to 6 mg 25-hydroxy vitamin D₃ (if 25-hydroxy vitamin D₃ is the sole vitamin D₃ compound), and further from 80,000 to 120,000 IU Vitamin A, from 1000 to 3000 IU Vitamin E, from 10 to 20 mg Biotin, from 200 to 300 mg copper (as CuSO₄), from 300 to 600 mg zinc (as ZnSO₄), from 5 to 10 mg cobalt (as CoSO₄), from 1 to 6 mg selenium (as Na₂SeO₃), from 5 to 10 mg iodine (as KI) and/or from 200 to 400 mg manganese (as MnSO₄) in addition to the magnesium chloride or sulphate.

For mature dairy cows preferable daily dosages of 25-hydroxy vitamin D₃ per cow—if 25-hydroxy vitamin D₃ is used as the sole vitamin D₃ compound—are in the range of from 1 to 10 mg, preferably from 1 to 6 mg, most preferred about 3 mg. If 1-α-hydroxy vitamin D₃ and/or 1-α-, 25-dihydroxy vitamin D₃ or mixtures of vitamin D₃ compounds containing one or both of these are used, the individual amount of each vitamin D₃ compound should be adapted accordingly.

Preferable daily dosages of one or more magnesium salts—preferably magnesium chloride and/or magnesium sulphate, most preferred magnesium chloride—per cow are in the range of from 50 to 150 g, preferably from 100 to 150 g, most preferred about 150 g.

According to the present invention it is preferred to start administering one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts, especially magnesium chloride and/or magnesium sulphate, most preferred magnesium chloride—one to four weeks prior to calving, most preferred approximately two weeks prior to calving.

The invention is further illustrated by the following examples.

### EXAMPLES

### Example 1

Four mature rumen fistulated cattle (approximately 600 kg liveweight) were held in individual pens and offered 12 kg/day of good quality lucerne chaff throughout the experiment. After a 2 week adaptation to this diet the following treatments were applied daily over 2 weeks to each animal in a Latin Square Design:

1. no supplement (control)

2. MgCl₂ (150 mg magnesium chloride),

3. ROVIMIX® Hy-D® 1.25% (5 mg 25-hydroxy vitamin D₃)

4. MgCl₂ plus ROVIMIX® Hy-D® 1.25%

experimental period. Blood samples were collected on days 7, 13 and 14 and urine samples on days 12, 13 and 14 of the period. Urine pH was measured at time of collection; blood plasma was decanted after centrifugation and analyzed for calcium, magnesium, potassium, sodium and phosphorus by inductively coupled plasma atomic emission spectrophotometry. The results were analysed as a latin square design making comparisons between treatments after removal of any animal or period effects. The lucerne chaff was assayed for macro cations together with chloride and sulphur concentrations.

The results are shown in Table 1.

The combination of magnesium chloride plus ROVIMIX® Hy-D® 1.25% resulted in an amplification in calcium and phosphorus mobilisation from bone mass that exceeds the enhancement observed when using magnesium chloride alone.

TABLE 1
Effects of MgCl₂ and Hy-D on urine pH, and urine and blood plasma minerals

| | Live- | Feed | | | | | | | | | | | |
|---|---|---|---|---|---|---|---|---|---|---|---|---|---|
| | weight | Intake | Urine | | Urine Composition (mg/L) | | | | Blood Plasma compostion (mg/L) | | | | |
| Treatment | (kg) | (kg/day) | pH | Ca²⁺ | Mg²⁺ | K⁺ | Na⁺ | Phosphorus | Ca²⁺ | Mg²⁺ | K⁺ | Na⁺ | Phosphorus |
| Control | 616 | 11.8 | 7.00a | 226a | 337 | 5702 | 849 | 119 | 81 | 19 | 228 | 2234 | 63 |
| Hy-D ® | 602 | 11.8 | 7.17a | 224a | 264 | 5023 | 538 | 112 | 79 | 18 | 230 | 2222 | 62 |
| Mg | 602 | 11.8 | 6.20b | 397b | 411 | 6652 | 444 | 190 | 87 | 19 | 233 | 2280 | 74 |
| Mg + Hy-D ® | 598 | 11.2 | 5.82b | 592c | 323 | 5167 | 379 | 244 | 77 | 17 | 217 | 2154 | 55 |
| Significance | NS | NS | P < 0.05 | P < 0.05 | NS | NS | NS | NS | NS | NS | NS | NS | NS |
| F Value | 1.24 | 2.28 | 4.86 | 8.64 | 1.5 | 1.25 | 2.41 | 0.66 | 0.75 | | 2.03 | 1.31 | 0.91 |
| 4.76 = 0.05 | | | | | | | | | | | | | |

### Example 2

A feed formulation for dry cows before calving containing 25-hydroxy vitamin D₃ and magnesium chloride can be prepared as follows (Dry Matter basis):

| Ingredients | % by weight |
|---|---|
| Maize silage | 30 |
| Grass silage | 20 |
| Cereal grain | 25 |
| Long hay | 15 |
| Protein meal | 8 |
| Vitamins and trace elements premix¹ | 2 |

¹containing the following per 100 g of premix:
3 mg ROVIMIX ® Hy-D ® 1.25%
100,000 IU Vitamin A,
2000 IU Vitamin E,
15 mg Biotin,
250 mg copper (as CuSO₄)
450 mg zinc (as ZnSO₄)
7.5 mg cobalt (as CoSO₄)
3 mg selenium (as Na₂SeO₃)
7.5 mg iodine (as KI),
300 mg manganese (as MnSO₄)
20 g magnesium (as MgCl₂)

The ingredients are mixed together.

### Example 3

A premix for a dairy cow food containing 25-hydroxy vitamin D₃ and magnesium chloride can be prepared as follows (active content in brackets):

| Ingredients | % by weight |
|---|---|
| ROVIMIX ® Hy•D ® 1.25% | 0.2 |
| Vitamin A 1000 | 0.05 |
| Vitamin E (50%) | 2 |
| Biotin (2%) | 0.375 |
| CuSO₄ (25%) | 0.5 |
| ZnSO₄ (80%) | 0.281 |
| CoSO₄ (20%) | 0.0185 |
| Na₂SeO₃ (44%) | 0.0035 |
| KI (68%) | 0.0055 |
| MnSO₄ (31%) | 0.484 |
| CaSO₄ | 21.08 |
| MgCl₂ | 75 |

All ingredients are carefully mixed together and 2% (2 kg/100 kg of food) of this premix is added to the dry cow food.

1. Use of one or more vitamin D₃ compounds chosen from the group 1-α-hydroxy vitamin D₃, 25-hydroxy vitamin D₃, 1-α-,25-dihydroxy vitamin D₃, in combination with one or more magnesium salts to prevent milk fever in animals.

2. Use of one or more vitamin D₃ compounds chosen from the group 1-α-hydroxy vitamin D₃, 25-hydroxy vitamin D₃, 1-α-,25-dihydroxy vitamin D₃, in combination with one or more magnesium salts for the manufacture of a food composition to prevent milk fever in animals.

3. Use according to claim 1, wherein 25-hydroxy vitamin D₃ is used as sole vitamin D₃ compound.

4. Use according to 1, wherein the magnesium salt is magnesium chloride and/or magnesium sulphate.

5. Use according to claim 2, wherein the food is a dry food composition which comprises from about 100 μg per kg to about 1000 μg per kg of 25-hydroxy vitamin D₃.

6. Use according to claim 1, wherein the animal is a dairy cow.

7. Method for preventing milk fever in animals said method comprising the step of administering to an animal in need of such treatment an amount of 1 mg to 10 mg of 25-hydroxy vitamin D₃ and from 50 g to 150 g of magnesium chloride and/or magnesium sulphate per day.

8. Method according to claim 7, wherein the animal is a dairy cow.

9. Method according to claim 7, wherein the administering starts one to four weeks prior to calving.

10. Food supplementation composition containing one or more vitamin D₃ compounds chosen from the group 1-α-hydroxy vitamin D₃, 25-hydroxy vitamin D₃, 1-α-, 25-dihydroxy vitamin D₃, in combination with one or more magnesium salts.
